(19) United States
(12) Patent Application Publication
(10) Pub. No.: US 2023/0073141 A1
(43) Pub. Date: Mar. 9, 2023

(54) AIR PURIFIER

(71) Applicant: Blueair AB, Englewood Cliffs, NJ (US)

(72) Inventors: Lars Henrik DUNBERGER, Stockholm (SE); Alagirisamy NETHAJI, Mumbai (IN); Johan Daniel WENNERSTRÖM, Sollentuna (SE)

(73) Assignee: Blueair AB, Englewood Cliffs, NJ (US)

(21) Appl. No.: 17/799,089

(22) PCT Filed: Feb. 10, 2021
(86) PCT No.: PCT/EP2021/053227

§ 371 Date: Aug. 11, 2022

(30) Foreign Application Priority Data
Feb. 14, 2020 (IN) 202021006449
Feb. 14, 2020 (IN) 202021006450
Feb. 14, 2020 (IN) 202021006451
Feb. 14, 2020 (IN) 202021006452
Feb. 14, 2020 (IN) 202021006453
Mar. 30, 2020 (EP) 20166875.3
May 26, 2020 (EP) 20176491.7

(51) Int. Cl. A61L 9/22; F24F 8/10; F24F 8/90

(52) U.S. Cl. CPC A61L 9/22; F24F 8/10; F24F 8/90; A61L 2209/14; A61L 2209/111

(57) ABSTRACT

A method for sterilising an internal surface of an air purifier or filter media in an air purifier by subjecting said internal surface or filter media to an ion cloud.

The present invention relates to an improved air purifier.

US 2007 034 082 discloses an air purifier including an ionizing assembly that operates to charge particulate material in an air flow passing through the purifier. The charged particulate material is attracted to and retained by a filter element disposed downstream of the ionization assembly and having an electrical charge opposite to the charged particulate material. The purified air passing through the filter is directed out of the device, optionally in conjunction with a fragrance that is added to the purified air flow. The ionizing assembly is formed with a ground member disposed adjacent the ionizing member to keep the electrons generated by the ionizing assembly within the purifier, and thus prevent static discharges from occurring outside of the purifier. The air flow is directed by a fan through the purifier in an angular and substantially laminar manner, such that the efficiency of the purifier is increased.

US 2002 141 131 discloses an improved air ionizer apparatus includes an air inlet, a high voltage source, an electrode electrically connected to the high voltage source for generating ions and an air outlet. An air mover is provided for causing air to flow into the air ionizer through the air inlet and out of the air ionizer through the air outlet. A foraminous filter comprising an electrically conductive material is electrically coupled to at least one of a voltage source and ground. The filter is positioned over at least one of the air inlet, the air outlet and the electrode, such that air flowing into the air inlet, air flowing out of the air outlet or air flowing past the electrode flows through the filter. In a preferred embodiment, the filter comprises a metal grid or screen.

WO 2018/058716 discloses an all-in-one fresh air purifier, comprising a housing (1), an indoor return air inlet (21), an outdoor fresh air outlet (22), a fresh air delivery outlet (2), an indoor return air discharge outlet (12) and a power supply control device provided in the housing (1); provided in sequence in the housing (1) are a stage one filter (3), a heat exchange core (4), an exhaust fan (5), a stage two filter (6), an ion cloud dust removal module, an air blower (9) and a stage three filter (10); the exhaust fan (5), ion cloud dust removal module and air blower (9) are electrically connected to the power supply control device. The present all-in-one device combines ventilation and air purification functionality, is plug-and-play, presents no difficulties in installation and maintenance or structural problems of installing air pipes and damaging a room, has high negative ion generation, has a long conveying distance, has strong dust removal and disinfection effects, and furthermore does not generate ozone during operation, maintaining a healthy environment.

CN 105 823 131 discloses a fresh air purification combined system for a teaching area. The fresh air purification combined system comprises an air treatment assembly. The air treatment assembly comprises a fresh air ventilator and an air purifier. The fresh air ventilator and the air purifier are arranged independently and fixed to different positions in a room, and thus the indoor air quality can be controlled through combination. According to the fresh air purification combined system for the teaching area, through the combination of the constant-oxygen air purifier and the purification type fresh air ventilator, a constant-clean, constant-oxygen, energy-saving and green ecological classroom can be created, and a safe and clean study environment is provided for students.

US 2005 055 990 discloses an air cleaner and method of controlling an operation of the same is disclosed. The air cleaner includes a cabinet having an inlet and an outlet; a filter assembly removing dust and smell particles from a room air; a fan; a sensor assembly sensing the composition of the room air; a supplier assembly providing at least one of insufficient components of the room air to a cleaned air; and a controller controlling the supplier assembly on the basis of data regarding the composition of the room air. In another aspect, the method of controlling the operation of the air cleaner includes steps of sensing a room air; measuring insufficient components of the room air and the amount thereof; and providing at least one of the insufficient components of the room air to a cleaned air.

Despite the prior art there remains a need for improved air purifiers, in particular air purifiers which are more hygienic throughout their working life.

Air purifiers work by filtering ambient air through a filter. Accordingly, anything in the air is in theory capturable by the filter. While there are different types of filtration means, from particulate filter to gas filter, it is a necessary consequence of the functioning of an air purifier that they also capture microbes caught in the air flow.

It is also a routine feature of air purifiers that the main focus is removing pollution from the ambient air and various sensors which indicate that particulates are being removed exist. Consequentially, it also is quite routine for an air purifier to work in an automatic mode whereby the presence of particulates influences the air flow speed through the device. Thus, when the air quality is good, it is possible and often desirable that the purifier is maintained in idle or stand-by mode to conserve energy.

However, when air is not passing through the purifier micro-organisms caught by the filter are able to grow quickly and form biofilms which can have a deleterious effect on effective filter life span and also cause a health hazard to the users who habit the domestic setting.

Accordingly, and in a first aspect there is provided a method for sterilising an internal surface of an air purifier or filter media in an air purifier by subjecting said internal surface or filter media to an ion cloud.

Preferably, the air purifier comprises an ioniser configured to create an ion cloud onto said internal surface or filter media.

The air purifier comprises an ioniser. Preferably, the ioniser comprises a corona discharge tip and a receiving electrode. When the corona discharge tip is subjected to an appropriate electric voltage it generates an ion cloud between the tip and the receiving or ground electrode.

The purifier may additionally comprise an ioniser disposed on the exterior of said purifier. Where an ioniser is disposed on the exterior of said device it is preferred that it is disposed at the top of the device. Locating the external ioniser at the top of the device means that domestic dust particles are ionised as they fall through the air towards the ground and are therefore more likely to aggregate as they become charged. As they become more aggregated they are more easily caught up in the air circulation pattern created by the device and so more easily filtered.

The ioniser disposed in the interior of the device is preferably located before the removable particulate filter in an air flow direction.

Preferably, the device comprises an interior ioniser and an exterior ioniser. The exterior ioniser facilitating aggregation of domestic dust particles and the interior ioniser facilitating capture of the aggregated dust particles by the removable particulate filter. In both instances the ionisation permits less dense filtration media and low air speed (fan) speeds.

The ioniser used comprises an emitter electrode and a ground electrode. The emitter electrode discharges an ion cloud between the emitter and the receiver on application of a suitable voltage, preferably from −10 to 10kV but more preferably around the range −8 to 8kV.

The emitter electrode is preferably a point, tip or multiple tips or points for example a brush and is in electrical communication with a voltage source.

The receiving electrode is preferably a metallic part in the shape of a ring so that the resulting ion cloud is in three dimensions as the ions are spent away from the emitter and are then pulled towards the receiver.

Preferably, the emitter is centrally disposed between a receiver in the form of a ring. The emitter may be disposed pointing towards or away from the air flow during use but it is preferred that the emitter is pointed towards the surface to be sterilised, for example, an internal wall or a filter medium.

In an alternatively preferred embodiment the ioniser is disposed on the removable filter medium. In such an embodiment the filter medium comprises at least the emitting electrode. The receiving electrode may be part of the purifier or part of the filter medium but in either case the removable filter medium comprises means for electrical communication with the purifier such that the ioniser may produce an ion filed when the filter medium is placed in a working configuration. In such an embodiment both the purifier and the filter comprise electrical connectors which co-operate to permit electricity to pass from the purifier to the ioniser disposed on the filter, i.e. both the emitter and the receiver.

Preferably, said ioniser comprises a corona discharge tip disposed within 15 cm from said surface to be sterilised.

In a second aspect there is provided a method for sterilising an internal surface or filter medium of an air purifier wherein said air purifier comprises an ioniser, a removable particulate or gas filter, an air flow generator, a means for controlling said air flow generator, a first air flow setting with an air filtration air flow speed and a second air flow setting which correlates with sterilisation of an internal surface of the air purifier and/or removable particulate or gas filter.

In a third aspect there is provided an air purifier comprising a removable filter medium, an air flow generator and an ioniser, said purifier comprising at least one air flow setting and a further setting where said ioniser is activated in the absence of air flow or in combination with an air flow setting such that said filter medium is subjected to from 0.1 to 1.2 cms⁻¹ media velocity air flow.

The air flow speed measured at the removable filter is known in the art as the media velocity. Media velocity is the velocity at which the air travels through the filter. Media velocity has to be controlled perfectly to ensure that the maximum amount of particles are trapped. Too fast and many of the pollutants fly straight through unfiltered. Too slow and the purifier is not reaching the farthest corners of your room quickly enough to be effective.

In a fourth aspect there is provided an air purifier comprising a removable filter medium, an air flow generator and an ioniser, a temperature sensor and a humidity sensor. We have surprisingly found that the air draft required to kill micro-organisms is significantly lower than that required for air filtration. Further, we have found that subjecting a surface to an air draft and then an ioniser, or vice versa provides enhanced sterilisation characteristics.

Preferably, the air purifier comprises removable filter media which may be particulate filter or gas filter.

Preferably, the air flow speed (media velocity) measured at the removable filter is at least 0.1 cms⁻¹ measured at the filter medium. The measurement at the filter medium is taken from the spatial centre point on the fan side of the filter media surface. Where there is more than one filter medium, the one taken for the air draft measurement is the one which is closest to the air flow generator and so receives the air draft first.

More preferably, the air flow speed measured at the removable filter is from 0.1 to 1.2 cms⁻¹. Most preferably, the air flow speed measured at the removable filter is from 0.2 to 1.1 cms⁻¹. Preferably, when in sterilisation mode the air flow generator generates an air flow commensurate with sterilisation of an internal surface of the air purifier and/or a filter media for a period of from 1 second to 10 hours. Similarly, when in sterilisation mode the air purifier generates an ion cloud for from 1 second to 10 hours either in the absence of any air flow or in the presence only of a low air speed such as would be measured at the filter medium at from 0.1 to 1.2 cms⁻¹.

Preferably, the ionisation (A) and low air speed air flow (0.1 to 1.2 cms^{-1,} (B)) are carried out exclusively, and more preferably sequentially. By this is meant that the surface to be sterilised is first subjected to an air draft and then when the air draft is stopped, the surface is exposed to a burst of ionisation. Similarly, the ionisation may come before the air draft.

It is also preferred that the sequence of air draft and then ionisation, or vice versa, is repeated such that the surface to be sterilised is subjected to both air draft and ionisation on an intermittent basis.

Preferably, (A) has a duration of from 1 min to 10 hours

Preferably, (B) has a duration of from 1 min to 10 hours.

In a preferred embodiment there is provided an air purifier comprising a removable particulate or gas filter, an air flow generator, a means for controlling said air flow generator, a first air flow setting with an air filtration air flow speed and a second air flow setting which correlates with sterilisation of an internal surface of the air purifier and/or removable particulate or gas filter and an ioniser.

Preferably, the air purifier comprises an operable mode for sterilising an internal surface or a filter medium and whereby the air draft corresponds with said second air flow setting and this is activated either before or after a period of ionisation. More preferably, said ionisation is conducted without any air flow or in conjunction with said second air flow setting.

We have surprisingly found that a combination of a modest air flow rate with ionisation treatment can prevent growth of micro-organisms and drastically reduce the prevalence of live micro-organisms being blown into the ambient air and also released into the environment during filter change.

Such micro-organisms include gram positive bacteria, gram negative bacteria, spores, moulds and fungi as well as any viruses within said micro-organisms.

We have surprisingly found that by providing a low speed air draft and then a burst of ionisation, or vice versa, we can drastically reduce micro-organism viability on the internal surfaces of the air purifier and in particular on the filter media. While the purifier is operating there is no increased risk of micro-organisms growing and multiplying on the filter. However, when it is in idle or stand-by mode and the conditions are favourable, the micro-organisms grow.

In preferred embodiments of the invention the purifier ascertains the likelihood of conditions conducive to micro-organism growth and when such conditions are deemed to exist it actuates the air flow generator and/or the ioniser to destroy the microbes on the filter, or even those on the internal surfaces of the purifier. Preferred means for assessing whether conditions are conducive to micro-organism propagation include humidity sensors and temperature sensors.

When the processor determines that the conditions are conducive to micro-organism growth it either provides an indication, for example by way of a visual or audible signal, or electronically to a remote device such as a mobile phone so that the user is notified that the air flow generator and/or ioniser should be employed or it automatically actuates the fan or impeller at a low speed as described herein and which is sufficient to prevent micro-organism growth or to directly destroy the micro-organisms.

Preferably, the purifier has first mode in which the choices are either: no action, where the conditions determined by the humidity sensor and temperature sensor are such that no or low micro-organisms growth is anticipated; an alert by way of an electronic signal to a mobile device to alert the user that conditions are favourable to micro-organisms and permitting the option for the user to actuate the fan; and a warning level where the user is warned that micro-organism growth is likely and strongly recommending to the user to actuate the fan or impeller.

A second mode may operate similarly in that indications are made determined by the input from the temperature and humidity sensors but instead of a warning or an alert, the machine is automatically turned on when conditions are such that micro-organism growth is likely.

The user of course may select one of these two modes where appropriate.

Temperature sensors are known in the art and are commercially available from Sensirion. Suitable examples of temperature sensors indude STS3x series.

Humidity sensors are known in the art and are commercially available from Sensirion. Suitable examples of humidity sensors include SHT3x series.

In a preferred embodiment the means for controlling the air flow generator based on input from said sensors is conducted automatically, for example by a processor. In such an embodiment the sensors sense the temperature and/or humidity on a continuous or intermittent basis and send information back to the processor. The processor determines whether the conditions are conducive to micro-organism growth based on at least temperature or humidity. Preferably, the processor determines whether the conditions are conducive to micro-organism growth based on temperature and humidity. More preferably, the processor determines the likelihood of micro-organism growth additionally based on parameters such as geographical location, time of the day, week, month or season or even the pollution levels as well as any specific conditions that occur, for example virus pandemics or bush fires, and combinations of any of these.

For example, in Asia, the wet seasons are typically defined by the monsoon and occurs in the summer. In contrast the summer in Europe and North America is characterised by drier weather. Similarly, the hemispheres have different seasonal characteristics.

Preferably, the geographical location is determined by GPS or through the purifiers WIFI capability. It may also be provided by way of user input during a set-up process.

The purifier is powered by any suitable power source induding internal sources, e.g. batteries, and external power sources. The power is used to drive a motor which in turn powers at least the air flow generator and the ioniser where present.

Preferably, the filter media comprises at least one of carbon, activated carbon, a non-woven, a thermoplastic, a thermosetting material, a porous foam, fibreglass, paper, a high loft spunbound web, a low loft spunbound web, a meltblown web and or a bi-modal fiber diameter meltblown media.

Preferably, the removable particulate filter is a High Efficiency Particulate Air (HEPA) filter. It is to be understood that while the filter part of an air purifier is a vital part of its function, air purifiers are not commonly manufactured with a filter in place. They are practically always manufactured separately and most importantly often by a different commercial enterprise than of the manufacturer of the air purifier itself. It is also typical for a manufacturer of filters to manufacture filters for different air purifier models made by different manufacturers. The particulate filter is to be contrasted with the pre-filter or any dust filter which is present. Preferably, the filter is pre-charged before application to the air purifier.

Pre-filters are filters which have a low air resistance and also function as a poke guard, preventing the user from touching the volute or impeller assembly. The pre-filters are not intended to exhibit any major effect in the context of air purification. They do not have the air resistance or particle entrainment capability of dedicated particulate filters. Preferably the pre-filter is not a HEPA filter.

The purifier of the inventions also comprises a fan or impeller. The fan may be a bladeless fan, an axial fan but it is preferred that the fan is a radial fan.

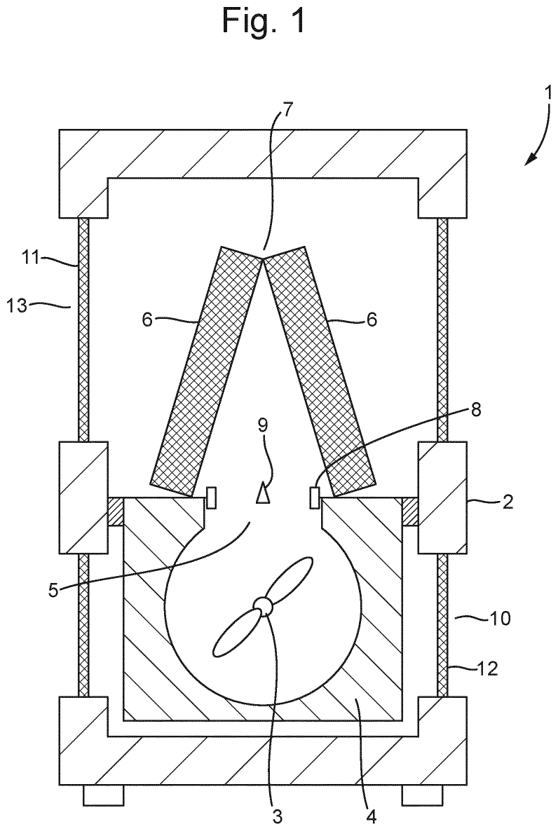

Embodiments of the invention will now be described with reference to the following in which FIG. 1 shows a cross section of an embodiment.

In detail, FIG. 1 shows an air purifier (1) comprising a housing (2) and a fan (3) contained in a volute (4). The fan (3) is shown in simplified form with no attempt made to describe its physical characteristics or placement. The volute (4) comprises an outlet (5) through which air is passed from the fan (3) to the filters (6). The filters (6) are connected at their top edges (7) to form an apex. The volute outlet (5) also comprises an ion emitter (9) and a ion receiver (8) for generating an ion filed (not shown) which extends towards the filters (6).

In use, air passes from ambient into the purifier through air inlets (10) which are secured with pre-filters (12) which act as an initial filter preventing large items entrained in the air flow from entering and blocking the internal mechanics of the device but also acting as a poke guard.

An air flow is then generated by the fan (3) and the air passes through the volute and towards the filters (6) where it is cleaned.

The air then passes out through the outlets (13). Similarly, the outlets (13) are also secured with a pre-filter (11).

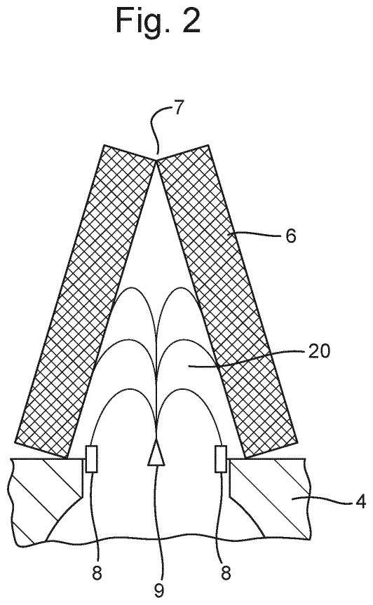

FIG. 2 is a schematic showing how the ion filed generated by the ioniser arrangement bathes the filters in an ion stream in use. Shown is the volute (4) and a corona discharge tip (9) which generates an ion stream between the tip (9) and the receiving electrodes (8) when an appropriate voltage is applied to the tip (9).

The ion cloud (20) extends away from the tip (9) and bathes the filters (6) and provides a sterilisation effect.

### EXAMPLE 1

The following experiment sets out to assess the impact of ionisation alone on micro-organism viability on a substrate, in this case a particulate filter. The ioniser was subjected to −5kV in order to emit an ion stream.

The air flow applied to the substrate in this experiment was zero.

The micro-organisms used were Staphylococcus aureus and Pseudomonas aeruginosa and the incubation period to generate the biofilm was 5 days.

Results:

| Exposure time (h) | 1 h | 2 h | 4 h |
|---|---|---|---|
| Log reduction microbes | 0.48 | 0.78 | 1.16 |

| Distance (cm) | 5 | 10 | 15 |
|---|---|---|---|
| Log reduction microbes | 0.75 | 0.5 | 0.28 |

Conclusion:

The closer the ioniser emitter to the substrate the better the impact on micro-organism viability. Further, a burst of ionisation around 2 hours performs most optimally.

1-7. (canceled)

8. A method for sterilizing filter media in an air purifier comprising a step of subjecting said filter media to an ion cloud wherein the air purifier comprises an ioniser configured to create an ion cloud onto said filter media and further wherein said ioniser comprises a corona discharge tip disposed within 15 cm from said filter media.

9. The method according to claim 8 wherein said air purifier comprises a removable particulate or gas filter, an air flow generator, a means for controlling said air flow generator, a first air flow setting with an air filtration air flow speed and a second air flow setting which correlates with sterilization of an internal surface of the air purifier and/or removable particulate or gas filter.

10. The method according to claim 9 wherein the air purifier comprises a removable filter medium, an air flow generator and an ioniser, said purifier comprising at least one air flow setting and a further setting wherein said ioniser is activated in the absence of air flow or in combination with an air flow setting and said filter medium is subjected to from 0.1 to 1.2 cms⁻¹ air flow.

11. An air purifier comprising a removable filter medium, an air flow generator and an ioniser configured to create an ion cloud onto said filter and wherein said ioniser comprises a corona discharge tip disposed within 15 cm from said filter media.

12. The air purifier according to claim 11 wherein the air purifier further comprises a temperature sensor and a humidity sensor.

13. The air purifier according to claim 11 wherein the air purifier captures microbes and pollution caught in air flow.

14. The air purifier according to claim 11 wherein the air purifier further comprises a fan or impellar.

15. The air purifier according to claim 14 wherein a fan is present and is a bladeless fan, axial fan or radial fan.

16. The air purifier according to claim 13 wherein the air purifier further comprises a sensor that indicates particulate is being removed from the air flow.
